(19) United States
(12) Patent Application Publication
(10) Pub. No.: US 2018/0360532 A1
(43) Pub. Date: Dec. 20, 2018

(54) METHOD AND SYSTEM FOR ABLATING A TISSUE

(71) Applicant: Lazcath Pty Ltd, Mosman Park (AU)

(72) Inventors: Pierre Jais, Mosman Park (AU); Peter Pratten, Mosman Park (AU); Rukshen Weerasooriya, Mosman Park (AU); Kamal Alameh, Mosman Park (AU)

(73) Assignee: Lazcath Pty Ltd, Mosman Park (AU)

(21) Appl. No.: 15/780,859

(22) PCT Filed: Dec. 5, 2016
(86) PCT No.: PCT/AU2016/051195

§ 371 Date: Jun. 1, 2018

(30) Foreign Application Priority Data
Dec. 3, 2015 (AU) 2015905025

(51) Int. Cl. A61B 18/14; A61B 18/18; A61B 18/24

(52) U.S. Cl. CPC A61B 18/1492; A61B 2018/00577; A61B 18/24; A61B 18/18

(57) ABSTRACT

A method of ablating a tissue comprising: (i) applying ablation energy to the tissue; (ii) applying light to the tissue and capturing an optical signal reflected therefrom; and (iii) monitoring the optical signal for a change indicative of an increased risk of a steam pop occurring in the tissue.

### FIELD OF THE INVENTION

The present invention relates to methods and systems for ablating tissue and to methods and systems that predict and/or otherwise assess the risk of a steam pop occurring in a tissue during ablation using an optical signal from the tissue.

### BACKGROUND TO THE INVENTION

Catheter ablation is a validated therapy for most arrhythmias including AF. However, the ablation process can cause undesirable charring of the tissue and localized coagulation, and can evaporate water in the blood and tissue leading to steam pops. The damage caused by steam pops can cause a number of problems due to the removal and ejection of tissue, and these problems can lead to stroke or death.

With the above in mind there is a need for improving the speed, safety and efficacy of catheter ablation by controlling ablation power and avoiding intra tissue steam “pop” occurrence which is detrimental to the ablation and potentially dangerous to the subject in the short term by over-penetration or “crater” and clot formation and in the medium term by altered scarring at the ablation site.

### SUMMARY OF THE INVENTION

According to a first aspect, the present invention provides a method of ablating a tissue comprising:
(i) applying ablation energy to the tissue;
(ii) applying light to the tissue and capturing an optical signal reflected therefrom; and
(iii) monitoring the optical signal for a change indicative of an increased risk of a steam pop occurring in the tissue.

According to a second aspect, the present invention provides a tissue ablation system comprising:
(i) a means for applying ablation energy to the tissue;
(ii) a means for applying light to the tissue and capturing an optical signal reflected therefrom; and
(iii) a processor for processing the optical signal to determine a change indicative of an increased risk of a steam pop occurring in the tissue.

According to a third aspect, the present invention provides a method for identifying increased risk of a steam pop occurring in a tissue subjected to ablation comprising:
(i) applying light to the tissue and capturing an optical signal reflected therefrom; and
(ii) monitoring the optical signal for a change indicative of an increased risk of the steam pop occurring in the tissue.

According to a fourth aspect, the present invention provides a tissue monitoring system comprising:
(i) a means for applying light to the tissue and capturing an optical signal reflected therefrom; and
(ii) a processor for processing the optical signal to determine a change indicative of an increased risk of a steam pop occurring in the tissue.

### BRIEF DESCRIPTION OF DRAWINGS

Preferred embodiments of the present invention are hereinafter described, by way of non-limiting examples only, with reference to the accompanying drawings, in which:

FIG. 1A is a representation of OCT images (from six sensors/channels) derived from a target muscle tissue to a depth 2.5 mm showing target tissue texture at time 15.24 seconds (at rest);

FIG. 1B is a representation of OCT images of the target muscle tissue in FIG. 1A at time 15.3 seconds (precursor to steam pop);

FIG. 1C is a representation of OCT images of the target muscle tissue in FIG. 1A at time 16.2 seconds (after the occurrence of a steam pop);

FIG. 1D is a representation of the concomitant sound wave associated with the steam pop at 16.08 seconds in the target muscle tissue, recorded in real time synchrony;

FIG. 2 is a schematic representation of the mechanism of a “Swept Laser Based” OCT that can be used in the method of the present invention or comprise a part of the system of the present invention;

FIG. 3 is a graph showing (i) the average amount of time (prediction time) between the method predicting a pop and the pop occurring (diamond line) and (ii) average amount of time taken for a pop to occur (average pop time) across three RF ablation powers tested with saline irrigation (square line);

FIG. 4a is an image of a single OCT channel showing changes in the signal that reflect changes in tissue texture due to an early stage of steam formation and FIG. 4b is a similar image showing the signal change prior to the occurrence of a pop;

FIG. 5 is an image of the signal of a single OCT channel composed of 240 A-lines with highlighted the region of interest of 100 A-lines;

FIG. 6a is an image of the signal of a single OCT channel showing the cross-correlation coefficient calculated between adjacent A-lines; and

FIG. 6b is an image of the signal of a single OCT channel showing the A-line partitioning for the calculation of the cross-correlation coefficients for each OCT channel.

### DETAILED DESCRIPTION OF THE INVENTION

According to a first aspect the present invention provides a method of ablating a tissue comprising:
(i) applying ablation energy to the tissue;
(ii) applying light to the tissue and capturing an optical signal reflected therefrom; and
(iii) monitoring the optical signal for a change indicative of an increased risk of a steam pop occurring in the tissue.

For the purposes of the present invention reference to “light” includes electromagnetic radiation or wave spectrum between ultraviolet and infrared.

For the purposes of the present invention the term “steam pop” includes the formation of steam in the tissue, the formation of microbubbles in the tissue and/or the formation of a steam pocket in the tissue

For the purposes of the present invention the term “tissue” includes organs such as the heart and parts thereof or parts within or adjacent thereto such as blood vessels including arteries and veins.

Preferably, the step of monitoring the optical signal is carried out in real time.

Preferably, the change indicative of an increased risk of a steam pop occurring in the tissue is indicative of change in tissue texture, form and/or structure. For example, said change may be a change in tissue texture at a point between the tissue surface and a tissue depth of at least 1, 2, 3, 4, or 5 mm.

More particularly, the change indicative of an increased risk of a steam pop occurring in the tissue may be a disruption in, loss of stability and/or consistency of the optical signal. More preferably, said change is a reduction or loss of linearity of the optical signal and/or an increase in the non-linearity of the optical signal. The change indicative of an increased risk of a steam pop may also be a change in an optical interference signal pattern. Such a change in the optical interference signal pattern may comprise at least one oblique, non-horizontal or irregular shaped band in said pattern.

Preferably, the increased risk comprises the statistical probability of a steam pop occurrence during the course of ablation.

Preferably, said change is indicative of an increased risk of a steam pop occurring at least 0.1-20 seconds, 0.2-15 seconds, 0.3-10 seconds, 0.3-15 seconds or at least 1, 2, 3, 4, 5, 6, 7, 8, 9, 10, 11, 12, 13 or 14 seconds after said change.

Preferably, the optical signal is monitored with or generated from a plurality of sensors such as, 2, 3, 4, 5, or 6 sensors. When the signal is monitored with or generated from a plurality of sensors it is preferred that the change indicative of an increased risk of a steam pop is present in the optical signal of at least 2 of the sensors.

Preferably the step of monitoring the optical signal comprises the step of combining the optical signal reflected from the tissue with a reference signal. Even more preferably the step of monitoring the optical signal comprises the step of conducting a reflected light wave interference analysis using the optical signal reflected from the tissue.

Preferably, the step of monitoring the optical signal comprises forming an electronic signal derived therefrom.

Preferably, the step of monitoring the optical signal comprises forming an image derived from the said optical signal.

Preferably, the optical signal is indicative of tissue information, such as tissue texture, form and/or structure, at a plurality of tissue depths therein or a portion thereof.

Preferably, the optical signal is represented as an image.

Preferably, the step of monitoring the optical signal comprises the step of comparing the optical signal from a location in the tissue at a first time point with the optical signal at the location at a second time point. In this form of the invention, it is preferred that the signals at the first and second time points are compared with each other or correlated to each other to determine if there has been a change in the signal between said time points. Even more preferably, the step of monitoring the optical signal comprises the step of comparing the optical signal from a location in the tissue at a first time point with the optical signal at the location at a second time point and the optical signal at the location at a third time point. In this regard, it is preferred to monitor the optical signal by comparing the optical signal from a location in the tissue with at least two adjacent time points to determine if there is a change indicative of an increased risk of a steam pop occurring in the tissue.

The optical signal may be presented as a plurality of axial lines or “A-lines” or a region of interest within said plurality of A-lines such as a region comprising a central portion of the plurality of the A-lines. For example, where an electronic signal is presented as an image comprising 240 A-lines, the region of interest may comprise a central portion of the 240 A-lines comprising about 50-150, 75-125 or 100 A-lines. In this regard, it has been found that a central portion of a larger image of an optical signal can be used to more accurately predict a pop.

Thus, the step of monitoring the optical signal may comprises the step of comparing the optical signal from a location in the tissue, as represented by a plurality, cluster or set of adjacent A-lines at a first time point with the optical signal at the location at a second time point. The plurality, cluster or set of adjacent A-lines may comprise 5, 10, 15, 20, 25, 30, 40 or 50 A-lines. Preferably, the method further comprises the step of determining the risk of a steam pop occurring and continuing or discontinuing the application of ablation energy to the tissue.

Preferably, the method further comprises the step of determining the risk of a steam pop occurring and continuing the application of ablation energy to the tissue albeit at a reduced energy level.

Preferably, the method further comprises the step of initiating or increasing tissue cooling, such as by irrigation or infusion, if the increased risk as reached a predetermined threshold level.

Preferably, the light is infra red or near infra red.

Preferably, the step of applying light to the tissue comprises applying the light to a depth of at least 1, 2, 3, 4, or 5 mm in the tissue.

Preferably, the step of applying light to the tissue comprises applying the light via a technique based on low-coherence interferometry. Even more preferably, the step of applying the light to the tissue comprises applying an optical tomographic technique such as an optical coherence tomographic technique.

Preferably, the step of capturing the optical signal comprises capturing an image from an interference signal pattern showing the tissue texture from surface to a depth of 3 mm.

Preferably at least one of steps (ii) and (iii) are carried out using optical coherence tomography.

Preferably, the ablation energy is heat energy, light energy, radio frequency energy, cryoenergy or ultra sound energy. When the ablation energy is light energy is it is preferably laser energy.

Preferably, the method is carried out over time to monitor the course of an ablation procedure.

According to a second aspect the present invention provides a tissue ablation system comprising:
(i) a means for applying ablation energy to the tissue;
(ii) a means for applying light to the tissue and capturing an optical signal reflected therefrom; and
(iii) a processor for processing the optical signal to determine a change indicative of an increased risk of a steam pop occurring in the tissue.

Preferably, the means for applying ablation energy to the tissue is a source of radio frequency energy, light energy, cryoenergy, radio frequency energy or ultra sound energy. Preferably, the means for applying ablation energy is a laser.

Preferably, the means for applying light is a laser. Preferably, the means for applying light to the tissue and capturing an optical signal reflected therefrom is an optical coherence tomography (OCT) system capable of generating tomographic data. In one particular form of the invention the means for applying light and the ablation energy is a multi-fibre optically switched fibre optic catheter.

When the means for applying light is an OCT system, the system may be configured to operate based on a frequency domain approach. Even more preferably, the system operates as a swept source OCT (SS-OCT). SS-OCT is adapted to perform a rapid, continuous sweep of the target tissue using a broad, longer wavelength optical imaging beam and can give improved visualisation of the target tissue including a greater depth of visualisation into the tissue e.g. 5-6 mm.

Preferably, the system includes a graphical display and/or an audio output (e.g., speaker) that provide visual and/or audio alarm when the system determines that the increased risk has reached a predetermined threshold level.

Preferably, the processor is adapted to control the means for applying ablation energy based on the optical signal or data derived therefrom such as the risk of a steam pop occurring in the tissue.

Preferably, the processor is adapted to initiate or increase tissue cooling, such as by irrigation or infusion, if the increased risk as reached a predetermined threshold level.

Preferably, the system further comprises a means for performing any of the method steps described with reference to the first aspect of the present invention.

In one specific form of the invention the system comprises one or more of the following features:
(i) a catheter for applying ablation energy to a tissue;
(ii) an array of switched focused multiple optical fibres incorporated in a catheter which is coupled to an OCT analyser controlled by a computer.
(iii) a catheter tip for emitting light that impinges on the tissue and captures light reflected by the tissue;
(iv) a controller, such as a computer, for the catheter;
(v) a fluid pumping system for delivering fluid to the tissue;
(vi) a processor for the reflected light;
(vii) a light source that delivers light to the catheter tip; and
(viii) a source of ablation energy.

Preferably, the catheter tip is for a first conduit for an optical imaging beam or light and a second conduit for an ablating means, the catheter tip being adapted to direct said beam and ablating means onto the tissue and capture a reflected portion of the optical imaging beam from the tissue portion.

Preferably, the catheter tip is for an array of first conduits such as 2-20 or 2-6 first conduits. In this regard, it is preferred that the catheter tip is for an array of light.

Preferably, the catheter tip comprises an optical directing component or lens for at least one of the said optical beams. Preferably, the optical directing component is multi-directional.

Preferably, the catheter tip further comprises a guidance system. The guidance system may be magnetic and thus the catheter tip may comprise at least one magnet. Preferably, the catheter tip comprises three magnets. Preferably, the magnets are located at or adjacent the leading end of the catheter tip.

The catheter tip may also comprise other guidance systems or components such as a tension wire or a coiled sheath guidance system. It will be appreciated that any guidance system may be incorporated into the catheter tip. Alternatively, the catheter tip may be provided without any specific guidance system.

According to a third aspect, the present invention provides a method for identifying increased risk of a steam pop occurring in a tissue subjected to ablation comprising:
(i) applying light to the tissue and capturing an optical signal reflected therefrom; and
(ii) monitoring the optical signal for a change indicative of an increased risk of the steam pop occurring in the tissue.

Other preferred aspects of this aspect of the invention are as described with reference to the first and second aspects of the invention.

According to a fourth aspect, the present invention provides a tissue monitoring system comprising:
(i) a means for applying light to the tissue and capturing an optical signal reflected therefrom; and
(ii) a processor for processing the optical signal to determine a change indicative of an increased risk of a steam pop occurring in the tissue.

Other preferred aspects of this aspect of the invention are as described with reference to the first and second aspects of the invention.

General

Each document, reference, patent application or patent cited in this text is expressly incorporated herein in their entirety by reference, which means that it should be read and considered by the reader as part of this text. That the document, reference, patent application or patent cited in this text is not repeated in this text is merely for reasons of conciseness. The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Those skilled in the art will appreciate that the invention described herein is susceptible to variations and modifications other than those specifically described. The invention includes all such variation and modifications. The invention also includes all of the steps and features referred to or indicated in the specification, individually or collectively and any and all combinations or any two or more of the steps or features.

The present invention is not to be limited in scope by any of the specific embodiments described herein. These embodiments are intended for the purpose of exemplification only. Functionally equivalent products and methods are clearly within the scope of the invention as described herein.

The invention described herein may include one or more range of values (e.g. size etc). A range of values will be understood to include all values within the range, including the values defining the range, and values adjacent to the range which lead to the same or substantially the same outcome as the values immediately adjacent to that value which defines the boundary to the range, provided such an interpretation does not read on the prior art.

Throughout this specification, unless the context requires otherwise, the word “comprise” or variations such as “comprises” or “comprising”, will be understood to imply the inclusion of a stated integer or group of integers but not the exclusion of any other integer or group of integers.

Other definitions for selected terms used herein may be found within the detailed description of the invention and apply throughout. Unless otherwise defined, all technical terms used herein have the same meaning as commonly understood to one of ordinary skill in the art to which the invention belongs.

### DESCRIPTION OF THE PREFERRED EMBODIMENTS/EXAMPLES

The present invention now will be described more fully hereinafter with reference to the accompanying Examples and Figures, in which preferred embodiments of the invention are described. This invention may, however, be embodied in many different forms and should not be construed as limited to the embodiments set forth herein; rather, these embodiments are provided so that this disclosure will be thorough and complete, and will fully convey the scope of the invention to those skilled in the art.

### Example 1

### Prediction of Pop Production During Tissue Ablation

Materials/Methods

A regular Thermocool™ catheter (Biosense Webster™) was used to create RF lesions on chicken hearts in a wet lab. An 8Fr prototype catheter equipped with optical coherence tomography (OCT) was placed next to the RF catheter to image the tissue in real time during RF delivery. A total of 60 RF lesions (20 at 30, 50 and 60 w) were conducted without irrigation.

Results

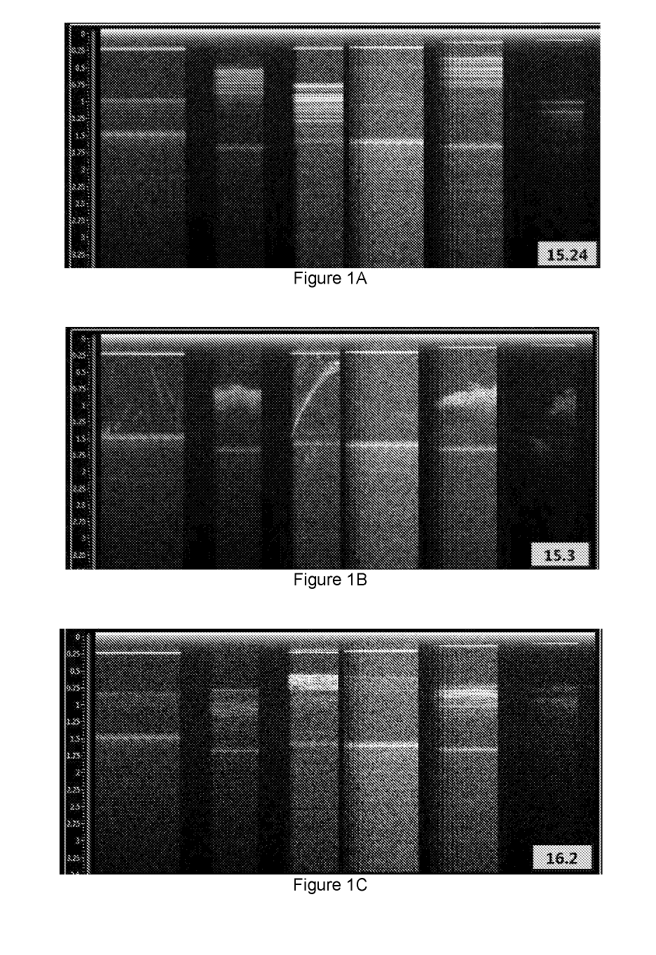

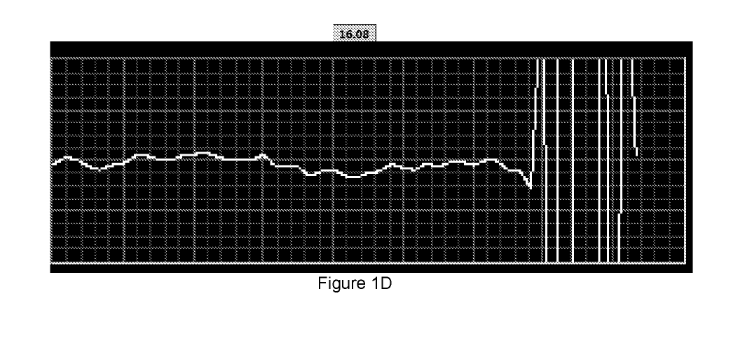

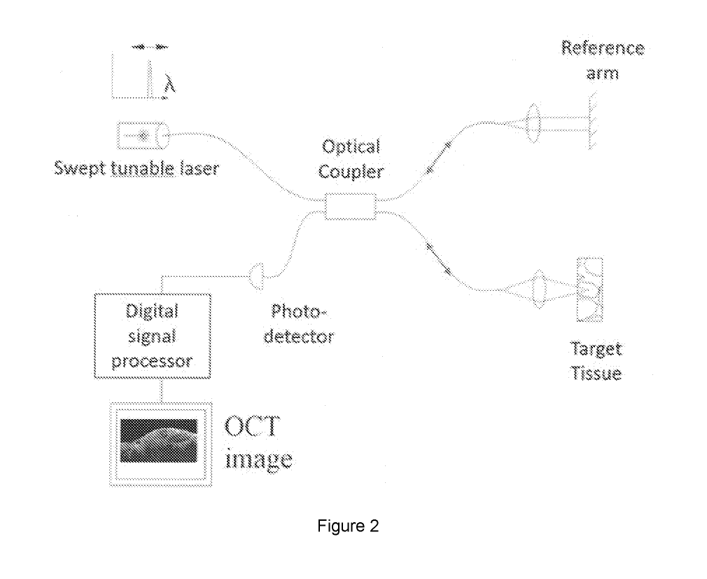

The results are depicted in FIGS. 1A-1C.

42 pops were observed (6@30, 17@50 and 19@60 W). Pops were always preceded by massive disruption of the OCT signal with loss of linearity as shown in the relevant figures. In particular, FIG. 1A at 15.24 sec, shows preserved linearity in all 6 sensors while 0.06 sec after, at 15.3 sec, linearity is lost in all sensors (see FIG. 1B. The pop is recorded on the audio track at 16.08 sec (see FIG. 1C).

These linearity losses on OCT appeared 0.3 to 10 seconds (1.35±2.27) prior to the pop at 50 W and 0 to 1.5 seconds (0.75±0.4) at 60 W. This warning delay accounted for 5%±1% of the RF time preceding the appearance of the linearity loss. None of the lesions without pops were associated with these specific & massive OCT changes.

### Example 2

### Pop Occurrence Prediction During Tissue Ablation

Methods

Experiments were conducted in a wet lab set up. Saline water was maintained at 37° C. with impedance between 100 and 150 ohms to mimic conditions in patients. Chicken heart ventricles were targeted with an irrigated ablation catheter, while the return pad was immersed in the bath. A weight of 15 g was attached on the shaft of the catheter to ensure constant contact force in the range of what occurs clinically.

An 8Fr OCT enabled catheter (Lazcath Pty Ltd—see for example the catheter arrangements in International patent application WO2016187664 incorporated herein by reference) including 6 OCT optical fibres was used and the signal displayed online on a computer screen. The OCT catheter was attached alongside to the RF catheter in order to achieve a fixed spatial relationship and be able to image the tissue with OCT as close as possible to the RF lesion.

The OCT catheter was calibrated before starting the experiments and OCT data were recorded continuously during RF deliveries.

RF was delivered at powers of 30, 40 and 50 W with irrigation (17 ml) for 60 sec or until a pop occurred. A waterproof microphone was placed in the bath to ensure accurate detection of pops. The audio signal was recorded on the OCT computer and displayed on the 6 channel OCT screen thereby allowing measurement of the delay between OCT first changes and the audio signal associated with the pop.

Results

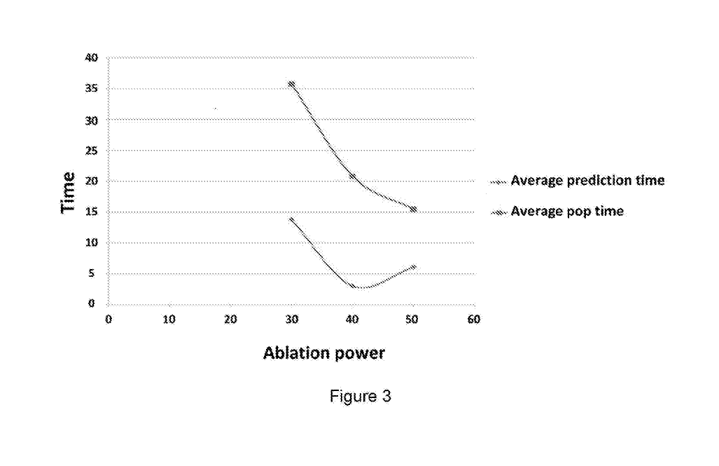

Table 1 and FIG. 3 include the results from this example.

TABLE 1

| RF Power | RF deliveries | Pops | Pops predicted | Success rate |
|---|---|---|---|---|
| 30 W | 50 | 7 | 7 | 100% |
| 40 W | 50 | 40 | 32 | 80% |
| 50 W | 50 | 48 | 36 | 75% |
| Totals | 150 | 95 | 75 | 79% |

As can be seen from FIG. 3, the average amount of time between the method predicting a pop and the pop occurring varied from about 14 seconds at 30 W to about 3 seconds at 40 W. Across the three power levels, of the pops predicted, the average prediction time was 7.7 seconds before their occurrence.

### Example 3

### OCT Signal Processing

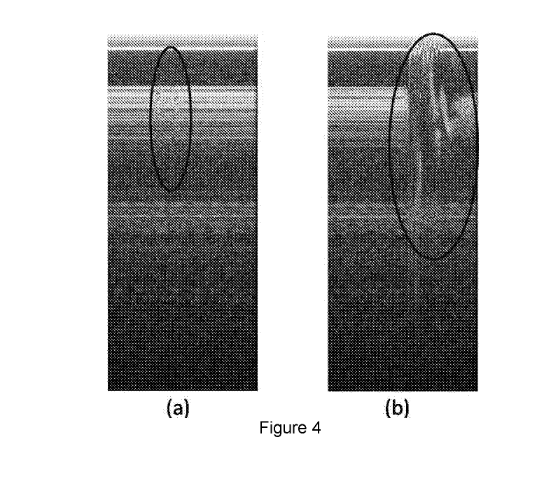

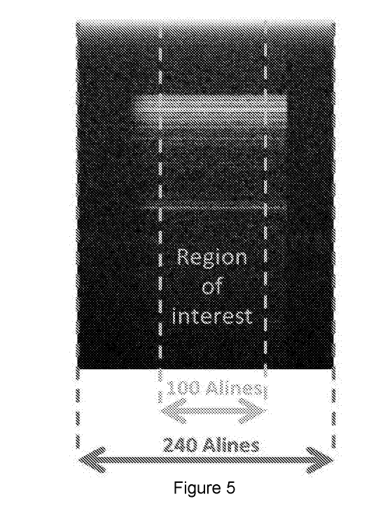

As described herein according to the present invention, applicants have noted that the OCT signal from a tissue undergoing ablation loses its stability and the tissue shows changes in texture ahead of the occurrence of a pop. FIG. 4 is another example showing these changes where FIG. 4a shows the changes in tissue texture/OCT signal due to an early stage of steam (pop) formation and FIG. 4b shows the changes in tissue texture/OCT signal just prior to the occurrence of a pop.

Described hereunder is one example of a method for processing an OCT signal to predict the occurrence (or risk thereof) of a pop. Once identified various visual and/or audio warnings can be generated to enable appropriate interventions to be initiated to avoid or reduce the risk of the pop occurring.

An algorithm was developed that calculates the cross-correlation between the axial lines (“A-lines”) that form an OCT image. This cross-correlation is a measure of the similarity between the consecutive OCT images.

Each OCT channel is made up of a certain number e.g. 240 A-lines and we found that it is preferred to base the signal processing on a region of interest in the central area of an OCT image—in this example a central region of about 100 A-lines was found to be particularly suitable for pop prediction.

Prior to a pop, the OCT images lose their texture and a degradation in tissue layer linearity is observed, leading to a reduction in the normalised cross-correlation coefficient between the A-lines.

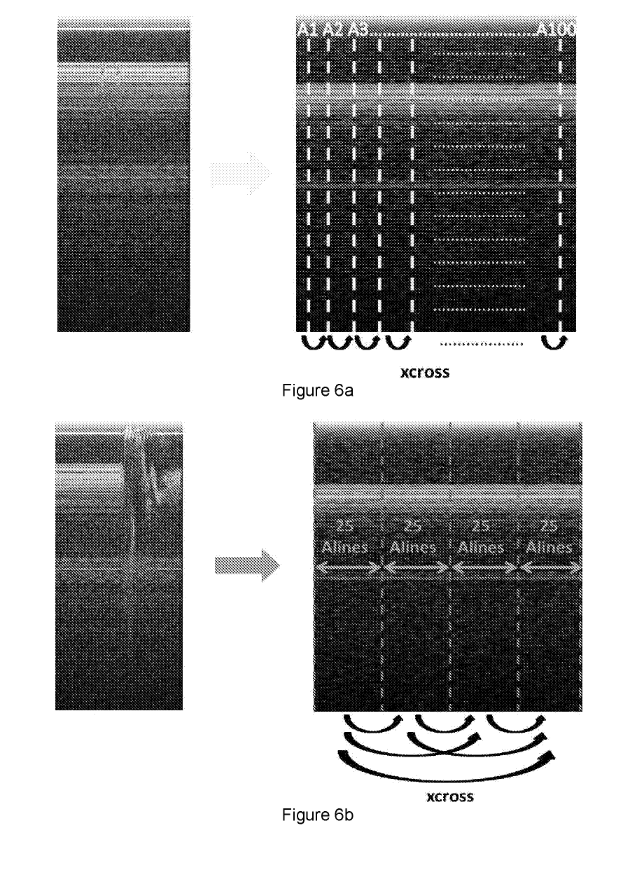

A pop warning signal can be triggered by either a single A-line or a group of A-lines, as illustrated in FIGS. 6a and 6b, respectively. In the first approach (FIG. 6a), the normalised cross-correlation coefficient is calculated between adjacent A-lines, i.e. each A-line is compared with the previous A-line and the following one. For the second approach (FIG. 6b), the central 100 A-lines of an OCT image are divided into 4 groups of 25 and the A-lines of each group were correlated with the corresponding A-lines of the other groups (FIG. 6b).

For each method a threshold is then defined according to Formula A:

Threshold=mean(xcross(i−1))×mean(xcross(i))×1−sdt(xcross(i))

Where i is the frame index and xcross is the normalized cross-correlation coefficient. When the cross-correlation coefficient of a frame is less than the threshold an audio and visual pop alarm is generated.

Based on the data herein, applicant was able to establish:
(i) definite reproducible change in the OCT character of the tissue prior to the production of the “pop”;
(ii) predict the onset of a “pop” prior to it occurring;
(iii) prevent the “pop” occurring by modulating the power of the ablation so the OCT display remained below this prediction level;
(iv) by varying the power of the ablation force in a sequential manner we enabled further optimisation of the power used for the ablation and at the same time prevented the occurrence of the “pop”.

The foregoing is illustrative of the present invention and is not to be construed as limiting thereof. Although a number of exemplary embodiments of this invention have been described, those skilled in the art will readily appreciate that many modifications are possible in the exemplary embodiments without materially departing from the novel teachings and advantages of this invention.

Accordingly, all such modifications are intended to be included within the scope of this invention as defined in the claims. Therefore, it is to be understood that the foregoing is illustrative of the present invention and is not to be construed as limited to the specific embodiments disclosed, and that modifications to the disclosed embodiments, as well as other embodiments, are intended to be included within the scope of the appended claims.

1. A method of ablating a tissue comprising:
(i) applying ablation energy to the tissue;
(ii) applying light to the tissue and capturing an optical signal reflected therefrom; and
(iii) monitoring the optical signal for a change indicative of an increased risk of a steam pop occurring in the tissue.

2. A method according to claim 1 wherein the change indicative of an increased risk of a steam pop occurring in the tissue is indicative of change in tissue texture, form and/or structure.

3. A method according to claim 1 or 2 wherein the change indicative of an increased risk of a steam pop occurring in the tissue is disruption in, loss of stability and/or consistency of the optical signal.

4. A method according to claim 3 wherein said change is a reduction or loss of linearity of the optical signal and/or an increase in the non-linearity of the optical signal.

5. A method according to claim 3 wherein the change is a change in the interference pattern of the optical signal.

6. A method according to claim 5 wherein the change in the interference pattern is at least one oblique, non-horizontal or irregular shaped band in said pattern.

7. A method according to any one of the preceding claims wherein said change is indicative of an increased risk of a steam pop occurring 0.1-20 seconds after said change.

8. A method according to any one of the preceding claims wherein the optical signal is monitored with or generated from a plurality of sensors.

9. A method according to claim 8 wherein the change indicative of an increased risk of a steam pop is present in the optical signal of at least two of the plurality of sensors.

10. A method according to claim 1 wherein the step of monitoring the optical signal comprises forming an electronic signal derived therefrom.

11. A method according to claim 1 wherein the step of monitoring the optical signal comprises forming an image derived from the said optical signal.

12. A method according to claim 1 wherein the optical signal is indicative of tissue information.

13. A method according to claim 13 wherein the tissue information is selected from the group comprising tissue texture, tissue form and/or tissue structure.

14. A method according to claim 12 or 13 wherein the tissue information pertains to a plurality of tissue depths.

15. A method according to claim 1 wherein the optical signal is represented as an image.

16. A method according to claim 1 wherein the step of monitoring the optical signal comprises the step of comparing the optical signal from a location in the tissue at a first time point with the optical signal at the location at a second time point.

17. A method according to claim 16 wherein the optical signals at the first and second time points are compared with each other or correlated to each other to determine if there has been a change in the signal between said time points.

18. A method according to claim 1 wherein the step of monitoring the optical signal comprises the step of comparing the optical signal from a location in the tissue at a first time point with the optical signal at the location at a second time point and the optical signal at the location at a third time point.

19. A method according to claim 18 wherein the step of monitoring the optical signal comprises the step of comparing the optical signal from a location in the tissue with at least two adjacent time points to determine if there is a change indicative of an increased risk of a steam pop occurring in the tissue.

20. A method according to any one of the preceding claims wherein the optical signal is presented as a plurality of axial lines or “A-lines”.

21. A method according to any one of the preceding claims wherein the optical signal is presented as a region comprising a central portion of a plurality of axial lines or “A-lines”.

22. A method according to any one of the preceding claims wherein the step of monitoring the optical signal comprises the step of comparing the optical signal from a location in the tissue, as represented by a plurality, cluster or set of adjacent A-lines at a first time point with the optical signal from the location in the tissue as represented by the plurality, cluster or set of adjacent A-lines at a second time point.

23. A method according to claim 22 wherein the plurality, cluster or set of adjacent A-lines comprise 5-50 A-lines.

24. A method according to any one of the preceding claims further comprising the step of determining the risk of a steam pop occurring and continuing or discontinuing the application of ablation energy to the tissue.

25. A method according to any one of claims 1 to 23 further comprising the step of determining the risk of a steam pop occurring and continuing the application of ablation energy to the tissue albeit at a reduced energy level.

26. A method according to any one of the preceding claims further comprising the step of initiating or increasing tissue cooling, such as by irrigation or infusion, if the increased risk as reached a predetermined threshold level.

27. A method according to any one of the preceding claims wherein the light is infra red or near infra red.

28. A method according to any one of the preceding claims wherein the step of applying light to the tissue comprises applying the light to a depth of at least 1-5 mm in the tissue.

29. A method according to any one of the preceding claims wherein the step of applying light to the tissue comprises applying the light via a technique based on low-coherence interferometry.

30. A method according to any one of the preceding claims wherein the step of applying the light to the tissue comprises applying an optical tomographic technique such as an optical coherence tomographic technique.

31. A method according to any one of the preceding claims wherein the step of capturing the optical signal comprises capturing an image from an interference signal pattern showing the tissue texture from surface to a depth of 3-6 mm.

32. A method according to claim 1 wherein at least one of steps (ii) and (iii) are carried out using optical coherence tomography.

33. A method according to any one of the preceding claims wherein the ablation energy is heat energy.

34. A method according to any one of the preceding claims wherein the ablation energy is light energy.

35. A method according to any one of the preceding claims carried out in real time.

36. A tissue ablation system comprising:
(i) a means for applying ablation energy to the tissue;
(ii) a means for applying light to the tissue and capturing an optical signal reflected therefrom; and
(iii) a processor for processing the optical signal to determine a change indicative of an increased risk of a steam pop occurring in the tissue.

37. A system according to claim 36 wherein the means for applying ablation energy to the tissue is a source of radio frequency energy or light energy.

38. A system according to claim 36 or 37 wherein the means for applying light to the tissue and capturing an optical signal reflected therefrom is an optical coherence tomography (OCT) system capable of generating tomographic data.

39. A system according to claim 36, 37 or 38 wherein the means for applying light to the tissue is an OCT system configured to operate as a swept source OCT (SS-OCT).

40. A system according to any one of claims 36-39 further comprising a graphical display and/or an audio output capable of providing an alarm when the system determines that the increased risk reaches a predetermined threshold level.

41. A system according to any one of claims 36-40 wherein the processor is adapted to control the means for applying ablation energy based on the optical signal or data derived therefrom such as the risk of a steam pop occurring in the tissue.

42. A system according to any one of claims 36-41 wherein the processor is adapted to initiate or increase tissue cooling, such as by irrigation or infusion, if the increased risk reaches a predetermined threshold level.

43. A system according to any one of claims 36-42 further comprising at least one of the following features:
(i) a catheter for applying ablation energy to a tissue;
(ii) an array of switched focused multiple optical fibres incorporated in a catheter which is coupled to an OCT analyser controlled by a computer.
(iii) a catheter tip for emitting light that impinges on the tissue and captures light reflected by the tissue;
(iv) a controller, such as a computer, for the catheter;
(v) a fluid pumping system for delivering fluid to the tissue;
(vi) a processor for the reflected light;
(vii) a light source that delivers light to the catheter tip; and
(viii) a source of ablation energy.

44. A method for identifying increased risk of a steam pop occurring in a tissue subjected to ablation comprising:
(i) applying light to the tissue and capturing an optical signal reflected therefrom; and
(ii) monitoring the optical signal for a change indicative of an increased risk of the steam pop occurring in the tissue.

45. A tissue monitoring system comprising:
(i) a means for applying light to the tissue and capturing an optical signal reflected therefrom; and
(ii) a processor for processing the optical signal to determine a change indicative of an increased risk of a steam pop occurring in the tissue.
